(19) United States
(12) Patent Application Publication
(10) Pub. No.: US 2006/0234205 A1
(43) Pub. Date: Oct. 19, 2006

(54) In vitro test system for predicting patient tolerability of therapeutic agents

(75) Inventors: Ying Cao, Fremont, CA (US); Kimberly Denis-Mize, Concord, CA (US); Susan Wilson, Alameda, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/073,374

(22) Filed: Mar. 3, 2005

Related U.S. Application Data
(60) Provisional application No. 60/646,095, filed on Jan. 21, 2005.
(60) Provisional application No. 60/585,980, filed on Jul. 7, 2004.
(60) Provisional application No. 60/550,868, filed on Mar. 5, 2004.

(51) Int. Cl. C12Q 1/00; G01N 33/567; G01N 33/53

(52) U.S. Cl. 435/4; 435/7.2

(57) ABSTRACT

Methods for predicting patient tolerability to therapeutic agents, such as cytokines, lymphokines and immunotoxins, are disclosed. The methods utilize an in vitro model of vascular leak syndrome (VLS) to assess the effect of the agent in question on the permeability of large proteins across confluent monolayers of endothelial cells (EC).

### CROSS-REFERENCE TO RELATED APPLICATIONS

This application claims benefit under 35 U.S.C. § 119(e) of provisional application 60/646,095 filed on Jan. 21, 2005, provisional application 60/585,980 filed on Jul. 7, 2004, and provisional application 60/550,868 filed on Mar. 5, 2004, which applications are hereby incorporated by reference in their entireties.

### TECHNICAL FIELD

The present invention pertains generally to in vitro assay methods. In particular, the invention relates to methods for predicting the ability of patients to tolerate particular therapeutic agents, including immunotherapeutic agents, such as IL-2 muteins.

### BACKGROUND

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al., N. Engl. J. Med. (1987) 316:889-897; Rosenberg, Ann. Surg. (1988) 208:121-135; Topalian et al., J. Clin. Oncol. (1988) 6:839-853; Rosenberg et al., N. Engl. J. Med. (1988) 319:1676-1680; and Weber et al., J. Clin. Oncol. (1992) 10:33-40). The anti-tumor activity of IL-2 has best been described in patients with metastatic melanoma and renal cell carcinoma using Proleukin®, a commercially available IL-2 formulation from Chiron Corporation, Emeryville, Calif. Other diseases, including lymphoma, also appear to respond to treatment with IL-2 (Gisselbrecht et al., Blood (1994) 83:2020-2022).

A number of other therapeutic agents have also been used to treat cancer due to their anti-tumor activity. Such agents include interleukins including IL-3, IL-4, interferon (IFN)-α, GM-CSF, anti-ganglioside antibodies, cyclosporin A, cyclophosphamide, mitomycin C, FK973, monocrotaline pyrrole and cytosine arabinoside; and a number of immunotoxins, such as immunotoxins constructed with ricin A chain (RTA), blocked ricin (blR), saporin (SAP), pokeweed antiviral protein (PAP) and Pseudomonas exotoxin (PE).

However, the therapeutic utility of many of these therapeutic agents is hampered by poor tolerability and toxicity. For example, high dose IL-2 immunotherapy is associated with severe toxicities manifest most notably by vascular leak syndrome (VLS), severe flu-like symptoms (fever, chills, vomiting), hypotension and neurological changes (see, for example, Duggan et al., J. Immunotherapy (1992) 12:115-122; Gisselbrecht et al., Blood (1994) 83:2081-2085; and Sznol and Parkinson, Blood (1994) 83:2020-2022). VLS is also observed when other chemotherapeutic agents are used, such as those discussed above. The mechanism of VLS may be due to endothelial damage mediated by interactions of activated PBMC with endothelial cells, production of cytokines, inflammatory mediators, or structural motifs inherent to the agent (reviewed in Baluna and Vitetta (1997) Immunopharmacology 37:117-132; Baluna, et al (1999) Proc. Natl. Acad. Sci. USA 96:3957-3962). Although the precise mechanism underlying such toxicity and VLS is unclear, accumulating data suggests that IL-2-induced natural killer (NK) cells trigger dose-limiting toxicities (DLT) as a consequence of overproduction of pro-inflammatory cytokines including IFN-γ, TNF-α, TNF-β, IL-1β, and IL-6 that activate monocytes/macrophages and induce nitric oxide (NO) production leading to subsequent damage of endothelial cells (Dubinett et al., 1994; Samlowski et al., 1995).

Several IL-2 mutants have been developed in order to overcome the toxicity exhibited by the native molecule. Examples of such mutants are described in commonly owned, copending U.S. Provisional Application Ser. No. 60/550,868, filed Mar. 5, 2004.

In vitro models have been developed in order to examine the mechanisms of VLS. For example, Damle and Doyle, J. Bacteriol. (1989) 142:2660-2669, describes an in vitro test system for examining the effects of IL-2-activated killer (IAK) lymphocytes and a number of lymphokines on the transendothelial macromolecular flux across endothelial cell monolayers. The system measures the flux of FITC-albumin across human umbilical endothelial cells. Kotasek et al., Cancer Res. (1988) 48:5528-5532 describes an in vitro assay for determining the mechanism of lymphokine-activated cell-mediated cytotoxicity also using endothelial cell monolayers. Lindstrom et al., Blood (1997) 90:2323-2334, pertains to an in vitro model for toxin-mediated VLS using human endothelial cells grown on microporous supports and cultured under low pressure in the presence or absence of ricin toxin A chain.

Although these test systems have been used to study the mechanisms of VLS, they have not heretofore been utilized to predict the tolerability by patients to various therapies, such as immunotherapies using modified lymphokines and chemotherapeutic immunotoxins.

### SUMMARY OF THE INVENTION

The present invention provides simple and efficacious in vitro assay methods for predicting the ability of patients to tolerate particular therapeutic agents, such immunotherapeutic agents, and hence the therapeutic utility of such molecules. The methods utilize an assay system that monitors the leakage of proteins through an endothelial cell monolayer as a predictor of tolerability following the therapy in question. The assay is particularly suited to predict tolerability in humans to various immunotherapies, as DLT in humans (fever/chills, VLS, and hypotension) all have derivative correlations with pro-inflammatory cytokine and nitric oxide (NO) production.

Accordingly, in one embodiment, the invention is directed to an in vitro method for predicting tolerability or intolerability by a patient to a selected therapeutic agent. The method comprises:

(a) providing a confluent monolayer of endothelial cells attached to an adherence substrate;

(b) contacting the monolayer with
(i) the selected therapeutic agent, or a preparation of lymphokine-activated killer (LAK) cells wherein the LAK cells are produced by activating peripheral blood mononuclear cells using the therapeutic agent, or the supernatant from the LAK cells, and
(ii) a detectably labeled macromolecule, wherein the detectably labeled macromolecule is substantially retained by the confluent monolayer when the monolayer is intact;

(c) incubating the monolayer from step (b) for a period of time and under conditions that allow for the detectably labeled macromolecule to pass through the confluent monolayer and the adherence substrate if the integrity of the monolayer is disrupted; and

(d) detecting macromolecule that passes through the confluent monolayer and the adherence substrate as an indication of tolerability or intolerability by a patient to the therapeutic agent.

In certain embodiments of the method, the therapeutic agent is an immunotherapeutic agent such as an IL-2 mutein, or an immunotoxin, or a small molecule chemotherapeutic agent.

In additional embodiments the adherence substrate used in the method comprises a collagen matrix.

In still further embodiments, the endothelial cells used in the method are human umbilical vein endothelial cells (HUVEC).

In other embodiments, the detectably labeled macromolecule used in the method is a detectably labeled albumin, such as a labeled bovine serum albumin (BSA). The BSA can be fluorescently labeled, such as with FITC.

In yet an additional embodiment, the invention is directed to an in vitro method for predicting tolerability or intolerability by a patient to an IL-2 mutein. The method comprises:

(a) providing a confluent monolayer of endothelial cells attached to an adherence substrate;

(b) contacting the monolayer with
(i) a preparation of lymphokine-activated killer (LAK) cells, wherein the LAK cells are produced by activating peripheral blood mononuclear cells using the IL-2 mutein, and
(ii) a detectably labeled macromolecule, wherein the detectably labeled macromolecule is substantially retained by the confluent monolayer when the monolayer is intact;

(c) incubating the monolayer from step (b) for a period of time and under conditions that allow for the detectably labeled macromolecule to pass through the confluent monolayer and the adherence substrate if the integrity of the monolayer is disrupted; and

(d) detecting macromolecule that passes through the confluent monolayer and the adherence substrate as an indication of tolerability or intolerability by a patient to the IL-2 mutein.

In certain embodiments the adherence substrate used in the method comprises a collagen matrix.

In further embodiments, the endothelial cells used in the method are human umbilical vein endothelial cells (HUVEC).

In other embodiments, the detectably labeled macromolecule used in the method is a detectably labeled albumin, such as a labeled BSA. The BSA can be fluorescently labeled, such as with FITC.

In an additional embodiment, the invention is directed to an in vitro method for predicting tolerability or intolerability by a patient to an IL-2 mutein. The method comprises:

(a) providing a confluent monolayer of human umbilical vein endothelial cells (HUVEC) attached to an adherence substrate comprising a collagen matrix;

(b) contacting the monolayer with
(i) a preparation of lymphokine-activated killer (LAK) cells, wherein the LAK cells are produced by activating peripheral blood mononuclear cells using the IL-2 mutein, and
(ii) a fluorescently labeled albumin;

(c) incubating the monolayer from step (b) for a period of time and under conditions that allow for the fluorescently labeled albumin to pass through the confluent monolayer and the adherence substrate if the integrity of the monolayer is disrupted; and

(d) detecting fluorescently labeled albumin that passes through the confluent monolayer as an indication of tolerability or intolerability by a patient to the IL-2 mutein.

In certain embodiments of the method, the fluorescently labeled albumin is BSA. The BSA can be fluorescently labeled, such as with FITC.

These and other embodiments of the subject invention will readily occur to those of skill in the art in view of the disclosure herein.

### BRIEF DESCRIPTION OF THE FIGURES

FIGS. 1A-1C depict the results of experiments conducted using 25 nM IL-2 mutein-stimulated LAK cells in the presence of supernatant from stimulated culture from three different subjects, respectively. Fluorescence intensity is used as a measure of migration of FITC-BSA across HUVEC monolayers following 22 hours of incubation with the LAK cells and supernatant. *: P value <0.1 vs medium control, t-Test: Paired Two sample for Means (n=5); $: P value <0.1 vs Proleukin, t-Test: Paired Two sample for Means (n=5); &: P value <0.1 vs F42E, t-Test: Paired Two sample for Means (n=5); @: P value <0.1 vs rIL-2, t-Test: Paired Two sample for Means (n=5).

FIGS. 2A-2C depict the results of experiments conducted using 25 nM IL-2 mutein-stimulated LAK cells from three different subjects, respectively. Fluorescence intensity is used as a measure of migration of FITC-BSA across HUVEC monolayers following 22 hours of incubation with the LAK cells without supernatant. *: P value <0.1 vs medium control, t-Test: Paired Two sample for Means (n=5); $: P value <0.1 vs Proleukin, t-Test: Paired Two sample for Means (n=5); &: P value <0.1 vs F42E, t-Test: Paired Two sample for Means (n=5); @: P value <0.1 vs rIL-2, t-Test: Paired Two sample for Means (n=5).

FIGS. 3A-3C show the results of experiments conducted using supernatant from 25 nM IL-2 mutein-stimulated LAK cells from three different subjects, respectively. Fluorescence intensity is used as a measure of migration of FITC-BSA across HUVEC monolayers following 22 hours of incubation with the supernatant. *: P value <0.1 vs medium control, t-Test: Paired Two sample for Means (n=5); $: P value <0.1 vs Proleukin, t-Test: Paired Two sample for Means (n=5); &: P value <0.1 vs F42E, t-Test: Paired Two sample for Means (n=5); @: P value <0.1 vs rIL-2, t-Test: Paired Two sample for Means (n=5).

FIGS. 4A-4C show the results of three independent experiments conducted using 25 nM IL-2 mutein. Fluorescence intensity is used as a measure of migration of FITC-BSA across HUVEC monolayers following 22 hours of incubation with the IL-2. *: P value <0.1 vs medium control, t-Test: Paired Two sample for Means (n=5); $: P value <0.1 vs Proleukin, t-Test: Paired Two sample for Means (n=5); &: P value <0.1 vs F42E, t-Test: Paired Two sample for Means (n=5); @: P value <0.1 vs rIL-2, t-Test: Paired Two sample for Means (n=5).

### DETAILED DESCRIPTION OF THE INVENTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

I. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a cell” includes two or more cells, and the like.

The term “comprising” encompasses “including” as well as “consisting” e.g., a composition “comprising” X may consist exclusively of X or may include something additional, for example X+Y.

The term “substantially” does not exclude “completely” e.g., a composition that is “substantially free” from Y may be completely free from Y. Where necessary, the word “substantially” may be omitted from the definition of the invention.

The term “derived from” is used herein to identify the original source of a molecule but is not meant to limit the method by which the molecule is made which can be, for example, by chemical synthesis or recombinant means.

The term “immunotherapeutic agent” is used herein to denote an agent that is an immunopotentiator or an immunosuppressant and is useful for treating cancer. Such agents include, without limitation, various cytokines and lymphokines, such as a number of interleukins, including IL-1, IL-2, IL-3, IL-4, IL-5, IL-12 and muteins of these molecules; interferons, such as but not limited to IFN-α, IFN-β, IFN-γ and muteins thereof; colony stimulating factors such as GM-CSF and muteins of GM-CSF; tumor necrosis factors, such as TNF-α and TNF-β and muteins of these molecules. Also captured by the term “immunotherapeutic agent” are immunotoxins. By “immunotoxin” is meant an antibody-toxin conjugate intended to destroy specific target cells (e.g., tumor cells) which bear antigens homologous to the antibody. Examples of toxins that are coupled to such antibodies include but are not limited to ricin A chain (RTA), blocked ricin (blR), saporin (SAP), pokeweed antiviral protein (PAP) and Pseudomonas exotoxin (PE), and other toxic compounds, such as radioisotopes and other chemotherapeutic drugs.

The term “IL-2” as used herein is a protein derived from a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5. Both full-length IL-2 proteins and biologically active fragments thereof are encompassed by the definition. The term also includes postexpression modifications of the IL-2, for example, glycosylation, acetylation, phosphorylation and the like.

The term “mutein” as used herein refers to a protein which includes modifications, such as deletions, truncations, additions and substitutions to the native sequence. Typically, the protein maintains biological activity, i.e., anti-tumor activity. These modifications may be deliberate, as through site-directed mutagenesis, or may be accidental, such as through mutations of hosts which produce the proteins or errors due to PCR amplification. The term “mutein” is used interchangeably with the terms “variant” and “analog.” The amino acid sequences of such muteins can have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. Methods for making polypeptide muteins are known in the art and are described further below.

Muteins will include substitutions that are conservative or non-conservative in nature. A conservative substitution is one that takes place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the protein of interest may include up to about 5-10 conservative or non-conservative amino acid substitutions, or even up to about 15-25, 50 or 75 conservative or non-conservative amino acid substitutions, or any integer between 5-75, so long as the desired function of the molecule remains intact. One of skill in the art can readily determine regions of the molecule of interest that can tolerate change.

The term “mutein” also refers to derivatives of the native molecule. By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, so long as the desired biological activity of the native polypeptide is retained. Methods for making polypeptide fragments, analogs, and derivatives are generally available in the art.

By “fragment” is intended a molecule consisting of only a part of the intact full-length sequence and structure. The fragment can include a C-terminal deletion an N-terminal deletion, and/or an internal deletion of the native polypeptide. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains biological activity, such as anti-tumor activity, as defined herein.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “cell culture” and “tissue culture” are used interchangeably and denote the maintenance of cells in vitro, in suspension culture in a liquid medium, or on a surface such as glass, plastic or agar or other suitable matrix provided with a liquid medium. In general, “cell culture” necessitates a medium that is buffered to maintain a constant suitable pH. Media used in cell culture are generally formulated to include an adequate supply of necessary nutrients and can be osmotically tailored to the particular cells being maintained, with temperature and gas phase also being controlled within suitable limits. Cell culture techniques are well known in the art. See, e.g., Morgan et al., Animal Cell Culture, BIOS Scientific Publishers, Oxford, UK (1993), and Adams, R. L. P. Cell Culture for Biochemists, Second Edition, Elsevier (1990).

The term “endothelial cell” is used herein to denote differentiated, squamous cells derived from the innermost layer of cells that lines the cavities of the heart and the blood and lymph vessels. Endothelial cells derive from the mesodermal embryonic cell layers. Examples of endothelial cells are provided below.

By “peripheral blood mononuclear cell” or “PBMC” is meant a population of cells isolated from peripheral blood of a mammal, such as a human, using, e.g., density centrifugation. Generally, a PBMC population includes mostly lymphocytes and monocytes and lacks red blood cells and most polymorphonuclear leukocytes and granulocytes.

“Passage” refers to the act of subculturing a cell population. A “subculture” refers to a cell culture established by the inoculation of fresh sterile medium with a sample from a previous culture. Each repeated subculture is counted as one passaging event.

A suitable “macromolecule” for use in the present assays is a macromolecule of a sufficient size such that it is substantially retained by a confluent monolayer unless the monolayer is disrupted, thereby enhancing the permeability of the macromolecule through the confluent monolayer. By “enhanced” permeability is meant that the macromolecule moves through the monolayer in greater amounts or at a faster rate as compared to the movement through the monolayer in the absence of the disrupting agent, i.e., LAK cells stimulated with an immunotherapeutic agent that causes vascular leak syndrome. Especially useful macromolecules include a serum albumin such as bovine serum albumin (BSA), ovalbumin, keyhole limpet hemocyanin, immunoglobulin molecules, thyroglobulin, and other proteins well known to those skilled in the art.

As used herein, the terms “label” and “detectable label” refer to a molecule capable of detection, including, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used under the invention include, but are not limited to, horse radish peroxidase (HRP), fluorescein compounds, such as fluorescein 5(6)-isothiocyanate or fluorescein isothiocyanate isomer I both known as “FITC”, rhodamine, dansyl, umbelliferone, dimethyl acridinium ester (DMAE), Texas red, luminol, NADPH and α-β-galactosidase.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

As explained above, immunotherapy, such as IL-2 therapy, is used to treat a variety of cancers, such as metastatic melanoma, renal cell carcinoma and lymphoma. However, treatment methods are hampered by severe toxicities that are associated with immunotherapies, such as vascular leak syndrome (VLS), severe flu-like symptoms (fever, chills, vomiting), hypotension, neurological changes and nitric oxide (NO) production, leading to subsequent damage of endothelial cells. Hence, a number of muteins of immunotherapeutic agents, such as lymphokine muteins, have been produced in hopes of circumventing the above side-effects. The present invention provides in vitro means of testing these muteins, as well as other therapeutic agents, for indications of toxicity.

In particular, the present invention is based on the discovery that in vitro methodology can be used to accurately and efficiently predict the ability of a patient to tolerate therapy using a particular IL-2 mutein without the common side-effects that can occur when a patient undergoes IL-2 immunotherapy. The assay methods use an in vitro endothelial permeability model to monitor leakage of macromolecules through confluent endothelial cell monolayers. The macromolecules used in the assay are normally retained by confluent monolayers, or leak only slightly. However, when the integrity of the monolayer is compromised, e.g., by exposure to a therapeutic agent that causes vascular leak syndrome, or exposure to LAK cells (or the supernatant thereof) that have been produced using a therapeutic agent that causes vascular leak syndrome or the like, increased transendothelial permeability to the macromolecule occurs.

The assay is particularly suited to predict tolerability in humans to various lymphokine-based immunotherapies, such as IL-2 immunotherapy, as DLT in humans (fever/chills, VLS, and hypotension) all have derivative correlations with pro-inflammatory cytokine and NO production.

A number of permeability assays are known in the art and can be used for testing therapeutic agents, such as IL-2 muteins. See, e.g., Damle and Doyle, J. Bacteriol. (1989) 142:2660-2669; Kotasek et al., Cancer Res. (1988) 48:5528-5532; Stone-Wolff et al., J. Exp. Med. (1984) 159:828; Lindstrom et al., Blood (1997) 90:2323-2334.

In general, these assays use adherence substrates with confluent monolayers that normally allow substances such as soluble nutrients, metabolites and hormonal factors to pass while substantially preventing cell migration therethrough, unless the integrity of the confluent monolayer is compromised. Confluent monolayers for use in the subject assays are typically generated from endothelial cells, such as vascular and lymph endothelial cells. Examples of such cells include, but are not limited to, human umbilical vein endothelial cells (HUVEC), readily obtained as described in, e.g., Jaffe et al., J. Clin. Invest. (1973) 52:2745; Stroncek et al., Arteriosclerosis (1986) 137:1735-1742 and commercially available from a number of suppliers such as the American Type Culture Collection (ATCC), Manassas, Va. (e.g., catalog number CRL-1730) and Cascade Biologics, Portland, Oreg.; WB572 cells (spontaneously transformed human saphenous vein smooth muscle cells); SV-E6 cells (human saphenous vein cells stably transfected with the E6 viral oncogene); A7R5 cells (spontaneously transformed rat thoracic aorta smooth muscle cells; ATCC); GH3B6 cells (rat pituitary cells; ATCC, Bethesda, Md.); PVEC cells (rat pulmonary vein endothelial cells; J. Tissue Culture Res. (1986) 10:9); CPA47 (bovine endothelial cells; ATCC CRL 1733); CPAE cells (bovine endothelial cells; ATCC CCL 209); EJG cells (bovine endothelial cells; ATCC CRL 8659); FBHE (bovine endothelial cells; ATCC CRL 1395); HW-EC-C cells (human endothelial cells; ATCC CRL 1730); and T/G HA-VSMC cells (human vascular smooth muscle cells; ATCC CRL 1999).

Prior to use in the subject assays, the cells are routinely passaged and cultured in suitable media, well known to those of skill in the art. For example, endothelial cells can be cultured in a commercially available medium, such as the RPMI-10AB medium as described in the examples; RPMI 1640 supplemented medium, as described in e.g., Damle and Doyle, J. Bacteriol. (1989) 142:2660-2669; M199 supplemented medium, as described in e.g., Lindstrom et al., Blood (1997) 90:2323-2334; Endothelial Growth Medium, EGM, available from Cambrex, Baltimore, Md., containing 2% fetal calf serum, and other tissue culture media, well known to those of skill in the art. Additional factors, such as endothelial cell growth factor (ECGF), heparin and the like can be used. See, e.g., Thornton et al., Science (1983) 222:623. The appropriate number of passages prior to use will vary and depends on the life expectancy of the cell line used. Generally, HUVEC are used at passes 2-20, more typically, passages 3-10, even more typically, at less than 5 passages, such as 2, 3, 4 passages, in the assay.

After the desired number of passages, approximately 1×10³ to 1×10⁸, preferably 1×10⁴ to 1×10⁷, such as 1×10⁵ to 1×10⁶ cells are added to an appropriate adherence substrate. Confluent monolayers are established by culturing for an appropriate amount of time, depending on the type of cells used. For example, HUVAC are typically incubated for approximately 1-7 days, generally 2-5 days, such as 1, 2, 3, 4, 5, 6 or 7 days, until a confluent monolayer is established. Confluency can be assessed using techniques well known in the art, such as by testing with crystal violet.

Appropriate substrates for use in the present assays include membranous supports, such as microporous, permeable films developed for tissue culture which generally permit the free permeation of substances such as soluble nutrients, metabolites and hormonal factors through the membrane while preventing cell migration therethrough. Adherence supports, for example, include dextran polymers, polyvinyl chlorides, polyglycolic acids, polylactic acids, polylactic coglycolic acids, and/or silicon. Typically, the substrate will also include collagen, fibrin, fibronectin, laminin, and/or hyaluronic acid. Such supports are well known in the art and are available from, for example, Costar (Cambridge, Mass.). One example is a Transwell™ support (e.g., TRANSWELL-COL PTFE membrane having a membrane thickness of 25-50 μm, pore sizes from 0.4-3.0 μm and which is treated with Type I and Type II collagen derived from bovine placentae). Such supports allow substances to pass through the monolayer from an upper chamber into a lower chamber where they can be collected and measured. However, any suitable permeable membrane support will find use with the present methods, so long as migration through the monolayer can be monitored.

Once confluent monolayers are established, a detectably labeled macromolecule is added to the monolayer (e.g., to the upper chamber of a transwell support) in the absence of the test substance in order to provide a background measurement. The detectably labeled macromolecule is one of a sufficient size to be substantially retained by the confluent monolayer unless the monolayer becomes leaky due to the presence of molecules that cause damage to the monolayer. As explained above, macromolecules typically used in the assays of the invention include serum albumin such as bovine serum albumin (BSA), ovalbumin, keyhole limpet hemocyanin, immunoglobulin molecules, thyroglobulin, and other proteins well known to those skilled in the art. After the labeled macromolecule is added, the assay is allowed to proceed from 15 minutes to several hours, such as from 30 minutes to 48 hours, preferably 1 hour to 24 hours, more preferably, 2 hours to 12 hours, such as 1 . . . 2 . . . 3 . . . 4 . . . 5 . . . 6 . . . 7 . . . 8 . . . 12 . . . 20 . . . 24 . . . 40 . . . 48 or more hours. Labeled molecule that passes through the monolayer and into, e.g., the bottom chamber, can be monitored, e.g., using a spectrophotometer, in order to provide a baseline measurement. If a fluorescent label is used, fluorescence can be measured using a spectrofluorometer and expressed as relative fluorescence units. The measurement can be qualitative or quantitative. For example, albumin clearance can be calculated using the formula: Albumin clearance (μl)=V_{L}×A/L where V_{L} is the volume in the bottom chamber at the time of sampling, A is the fluorescence units/μl in the bottom chamber at the time of sampling and L is the fluorescence units/μl in the top chamber at the beginning of the assay. The rate of albumin clearance (μl /min) can be calculated by linear regression analysis. For example, the clearance (μl±SEM/min) can be determined.

After appropriate measurements are made, any labeled macromolecule remaining is removed and the test compounds are added, e.g., to the top wells, along with media containing the labeled macromolecule. For example, the therapeutic agent of interest, or LAK cells (or supernatant therefrom) that have been stimulated using the therapeutic agent in question are added, as well as controls, as detailed below. LAK cells are produced by activating peripheral blood mononuclear cells (PBMCs) using the selected therapeutic agent to activate the PBMCs. PBMCs for activation can be isolated from whole blood using techniques well known in the art, such as by using Ficoll-Hypaque density gradients. After centrifugation, adherent mononuclear cells can be, but need not be, separated from nonadherent mononuclear cells (NAMNC) by successive cycles of adherence to plastic for, e.g., 45 min. at 37 degrees C. In order to prepare activated cells, the therapeutic agent in question and PBMCs are combined. The amount of agent to be added will depend on the particular substance being tested. Thus, for example, when a lymphokine such as an IL-2 mutein is being assayed, the mutein is added at a concentration of 10-500 nM, generally at a concentration of 25-250 nM, even more preferably at a concentration of 35-100 nM. One of skill in the art can easily determine the appropriate concentration for use. See, e.g., Damle and Doyle, J. Bacteriol. (1989) 142:2660-2669; Damle et al., J. Immunol. (1987) 138:1779; Damle and Doyle, Int. J. Cancer (1987) 40:519; Damle et al., J. Immunol. (1986) 137:2814.

The assay is allowed to proceed as described above with respect to the baseline measurement. Samples can be removed from the bottom chamber at multiple time points and assessments of labeled macromolecule present can be performed. Various controls can be run, as detailed in the examples below. Particularly, therapeutic agents with improved tolerability can be used as negative controls to establish baseline leakage that occurs in the absence of a therapeutic agent that causes VLS. For example, IL-2 muteins F42E and Y107R are substitution mutants that exhibit increased tolerability. See, commonly owned, copending U.S. Provisional Application Ser. No. 60/550,868, filed Mar. 5, 2004. These muteins also maintain effector function in in vitro and in vivo models in terms of NK and T cell proliferation, as well as NK/LAK/ADCC activity. In addition, positive controls can be used, such as detergents and the like known to damage cell monolayers, for example, saponin. Medium controls can also be used.

As explained above, the methods of the present invention are used to test therapeutic agents such as IL-2 muteins for patient tolerability. A number of IL-2 muteins are known and described further below. However, the present methods are equally applicable to other IL-2 muteins not specifically described herein. Such IL-2 muteins can be derived from IL-2 obtained from any species. Such variants should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein. Suitable biologically active muteins of native or naturally occurring IL-2 can be fragments, analogs, and derivatives of that polypeptide, as defined above.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor Laboratory Press, Plainview, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

Guidance as to regions of the IL-2 protein that can be altered either via residue substitutions, deletions, or insertions can be found in the art. See, for example, the structure/function relationships and/or binding studies discussed in Bazan (1992) Science 257:410-412; McKay (1992) Science 257:412; Theze et al. (1996) Immunol. Today 17:481-486; Buchli and Ciardelli (1993) Arch. Biochem. Biophys. 307:411-415; Collins et al. (1988) Proc. Natl. Acad. Sci. USA 85:7709-7713; Kuziel et al. (1993) J. Immunol. 150:5731; Eckenberg et al. (1997) Cytokine 9:488-498; the contents of which are herein incorporated by reference in their entirety.

In constructing variants of the IL-2 polypeptide of interest, modifications are made such that muteins continue to possess the desired activity. Obviously, any mutations made in the DNA encoding the variant polypeptide must not place the sequence out of reading frame and preferably will not create complementary regions that could produce secondary mRNA structure. See EP Patent Application Publication No. 75,444.

Biologically active muteins of IL-2 will generally have at least about 70%, preferably at least about 80%, more preferably at least about 90% to 95% or more, and most preferably at least about 98%, 99% or more amino acid sequence identity to the amino acid sequence of the reference IL-2 polypeptide molecule, such as native human IL-2, which serves as the basis for comparison. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2:482-489. A variant may, for example, differ by as few as 1 to 15 amino acid residues, as few as 1 to 10 residues, such as 6-10, as few as 5, as few as 4, 3, 2, or even 1 amino acid residue.

With respect to optimal alignment of two amino acid sequences, the contiguous segment of the variant amino acid sequence may have the same number of amino acids, additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm). A biologically active variant of a native IL-2 polypeptide of interest may differ from the native polypeptide by as few as 1-15 amino acids, as few as 1-10, such as 6-10, as few as 5, as few as 4, 3, 2, or even 1 amino acid residue.

The precise chemical structure of a polypeptide having IL-2 activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as the IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of IL-2 polypeptides of interest as used herein.

The art provides substantial guidance regarding the preparation and use of polypeptide variants. In preparing the IL-2 muteins, one of skill in the art can readily determine which modifications to the native protein nucleotide or amino acid sequence will result in a variant that is suitable for use as a therapeutically active component of a pharmaceutical composition used in the methods of the present invention.

The IL-2 muteins for use in the methods of the present invention may be from any source, but preferably are recombinantly produced. By “recombinant IL-2” or “recombinant IL-2 mutein” is intended interleukin-2 or variant thereof that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for the IL-2 in question is cloned and then expressed in transformed organisms, preferably a microorganism. The host organism expresses the foreign gene to produce the IL-2 mutein under expression conditions. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

For examples of IL-2 muteins, see European Patent (EP) Publication No. EP 136,489 (which discloses one or more of the following alterations in the amino acid sequence of naturally occurring IL-2: Asn26 to Gln26; Trp121 to Phe121; Cys58 to Ser58 or Ala58, Cys105 to Ser105 or Ala105; Cys125 to Ser125 or Ala125; deletion of all residues following Arg 120; and the Met-1 forms thereof); and the recombinant IL-2 muteins described in European Patent Application No. 83306221.9, filed Oct. 13, 1983 (published May 30, 1984 under Publication No. EP 109,748), which is the equivalent to Belgian Patent No. 893,016, and commonly owned U.S. Pat. No. 4,518,584 (which disclose recombinant human IL-2 mutein wherein the cysteine at position 125, numbered in accordance with native human IL-2, is deleted or replaced by a neutral amino acid; alanyl-ser125-IL-2; and des-alanayl-ser125-IL-2). See also U.S. Pat. No. 4,752,585 (which discloses the following IL-2 muteins: ala104 ser125 IL-2, ala104 IL-2, ala104 ala125 IL-2, val104 ser125 IL-2, val104 IL-2, val104 ala125 IL-2, des-ala1 ala104 ser125 IL-2, des-ala1 ala104 IL-2, des-ala1 ala104 ala125 IL-2, des-ala1 val104 ser125 IL-2, des-ala1 val104 IL-2, des-ala1 val104 ala125 IL-2, des-ala1 des-pro2 ala104 ser125 IL-2, des-ala1 des-pro2 ala104 IL-2, des-ala1 des-pro2 ala104 ala125 IL-2, des-ala1 des-pro2 val104 ser125 IL-2, des-ala1 des-pro2 val104 IL-2, des-ala1 des-pro2 val104 ala125 IL-2, des-ala1 des-pro2 des-thr3 ala104 ser125 IL-2, des-ala1 des-pro2 des-thr3 ala104 IL-2, des-ala1 des-pro2 des-thr3 ala104 ala125 IL-2, des-ala1 des-pro2 des-thr3 val104 ser125 IL-2, des-ala1 des-pro2 des-thr3 val104 IL-2, des-ala1 des-pro2 des-thr3 val104 ala125 IL-2, des-ala1 des-pro2 des-thr3 des-ser4 ala104 ser125 IL-2, des-ala1 des-pro2 des-thr3 des-ser4 ala104 IL-2, des-ala1 des-pro2 des-thr3 des-ser4 ala104 ala125 IL-2, des-ala1 des-pro2 des-thr3 des-ser4 val104 ser125 IL-2, des-ala1 des-pro2 des-thr3 des-ser4 val104 IL-2, des-ala1 des-pro2 des-thr3 des-ser4 val104 ala125 IL-2, des-ala1 des-pro2 des-thr3 des-ser4 des-ser5 ala104 ser125 IL-2, des-ala1 des-pro2 des-thr3 des-ser4 des-ser5 ala104 IL-2, des-ala1 des-pro2 des-thr3 des-ser4 des-ser5 ala104 ala125 IL-2, des-ala1 des-pro2 des-thr3 des-ser4 des-ser5 val104 ser125 IL-2, des-ala1 des-pro2 des-thr3 des-ser4 des-ser5 val 104 IL-2, des-ala1 des-pro2 des-thr3 des-ser4 des-ser5 val104 ala125 IL-2, des-ala1 des-pro2 des-thr3 des-ser4 des-ser5 des-ser6 ala104 ala125 IL-2, des-ala1 des-pro2 des-thr3 des-ser4 des-ser5 des-ser6 ala104 IL-2, des-ala1 des-pro2 des-thr3 des-ser4 des-ser5 des-ser6 ala104 ser125 IL-2, des-ala1 des-pro2 des-thr3 des-ser4 des-ser5 des-ser6 val104 ser125 IL-2, des-ala1 des-pro2 des-thr3 des-ser4 des-ser5 des-ser6 val104 IL-2, and des-ala1 des-pro2 des-thr3 des-ser4 des-ser5 des-ser6 val104 ala125 IL-2) and U.S. Pat. No. 4,931,543 (which discloses the IL-2 mutein des-alanyl-1, serine-125 human IL-2, as well as the other IL-2 muteins).

Also see European Patent Publication No. EP 200,280 (published Dec. 10, 1986), which discloses recombinant IL-2 muteins wherein the methionine at position 104 has been replaced by a conservative amino acid. Examples include the following muteins: ser4 des-ser5 ala104 IL-2; des-ala1 des-pro2 des-thr3 des-ser4 des-ser5 ala104 ala125 IL-2; des-ala1 des-pro2 des-thr3 des-ser4 des-ser5 glu104 ser125 IL-2; des-ala1 des-pro2 des-thr3 des-ser4 des-ser5 glu104 IL-2; des-ala1 des-pro2 des-thr3 des-ser4 des-ser5 glu104 ala125 IL-2; des-ala1 des-pro2 des-thr3 des-ser4 des-ser5 des-ser6 ala104 ala125 IL-2; des-ala1 des-pro2 des-thr3 des-ser4 des-ser5 des-ser6 ala104 IL-2; des-ala1 des-pro2 des-thr3 des-ser4 des-ser5 des-ser6 ala104 ser125 IL-2; des-ala1 des-pro2 des-thr3 des-ser4 des-ser5 des-ser6 glu104 ser125 IL-2; des-ala1 des-pro2 des-thr3 des-ser4 des-ser5 des-ser6 glu104 IL-2; and des-ala1 des-pro2 des-thr3 des-ser4 des-ser5 des-ser6 glu104 ala125 IL-2. See also European Patent Publication No. EP 118,617 and U.S. Pat. No. 5,700,913, which disclose unglycosylated human IL-2 muteins bearing alanine instead of methionine as the N-terminal amino acid as found in the native molecule; an unglycosylated human IL-2 with the initial methionine deleted such that proline is the N-terminal amino acid; and an unglycosylated human IL-2 with an alanine inserted between the N-terminal methionine and proline amino acids.

Other IL-2 muteins include those disclosed in WO 99/60128 (substitutions of the aspartate at position 20 with histidine or isoleucine, the asparagine at position 88 with arginine, glycine, or isoleucine, or the glutamine at position 126 with leucine or gulatamic acid), which reportedly have selective activity for high affinity IL-2 receptors expressed by cells expressing T cell receptors in preference to NK cells and reduced IL-2 toxicity; the muteins disclosed in U.S. Pat. No. 5,229,109 (substitutions of arginine at position 38 with alanine, or substitutions of phenylalanine at position 42 with lysine), which exhibit reduced binding to the high affinity IL-2 receptor when compared to native IL-2 while maintaining the ability to stimulate LAK cells; the muteins disclosed in International Publication No. WO 00/58456 (altering or deleting a naturally occurring (x)D(y) sequence in native IL-2 where D is aspartic acid, (x) is leucine, isoleucine, glycine, or valine, and (y) is valine, leucine or serine); the IL-2 p1-30 peptide disclosed in International Publication No. WO 00/04048 (corresponding to the first 30 amino acids of IL-2, which contains the entire α-helix A of IL-2 and interacts with the b chain of the IL-2 receptor); and a mutant form of the IL-2 p1-30 peptide also disclosed in WO 00/04048 (substitution of aspartic acid at position 20 with lysine).

Additional examples of IL-2 muteins with predicted reduced toxicity are disclosed in U.S. Provisional Application Ser. No. 60/550,868, filed Mar. 5, 2004, herein incorporated by reference in its entirety. These muteins comprise the amino acid sequence of mature human IL-2 with a serine substituted for cysteine at position 125 of the mature human IL-2 sequence and at least one additional amino acid substitution within the mature human IL-2 sequence such that the mutein has the following functional characteristics: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2 under comparable assay conditions. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L361, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M461, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q126I, and Q126V; where the amino acid residue position is relative to numbering of the mature human IL-2 amino acid sequence. In other embodiments, these muteins comprise the amino acid sequence of mature human IL-2 with an alanine substituted for cysteine at position 125 of the mature human IL-2 sequence and at least one additional amino acid substitution within the mature human IL-2 sequence such that the mutein has these same functional characteristics. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K351, K35L, K35M, K35N, K35P, K35Q, K35T, L36A, L36D, L36E, L36F, L36G, L36H, L361, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M461, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q1261, and Q126V; where the amino acid residue position is relative to numbering of the mature human IL-2 amino acid sequence. In alternative embodiments, these muteins comprise the amino acid sequence of mature human IL-2 with at least one additional amino acid substitution within the mature human IL-2 sequence such that the mutein has these same functional characteristics. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K351, K35L, K35M, K35N, K35P, K35Q, K35T, L36A, L36D, L36E, L36F, L36G, L36H, L361, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M461, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L941, L94T, L94V, L94Y, E95D, E95G, E95M, T102S, T102V, M104G, E106K, Y107H, Y107K, Y107L, Y107Q, Y107R, Y107T, E116G, N 119Q, T123S, T123C, Q126I, and Q126V; where the amino acid residue position is relative to numbering of the mature human IL-2 amino acid sequence. Additional muteins disclosed in U.S. Provisional Application Ser. No. 60/550,868 include the foregoing identified muteins, with the exception of having the initial alanine residue at position 1 of the mature human IL-2 sequence deleted.

The IL-2 mutein may also be an IL-2 fusion or conjugate comprising IL-2 fused to a second protein or covalently conjugated to polyproline or a water-soluble polymer to reduce dosing frequencies or to improve IL-2 tolerability. For example, the IL-2 mutein can be fused to human albumin or an albumin fragment using methods known in the art (see WO 01/79258). Alternatively, the IL-2 mutein can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

The present assay methods are also useful for testing other therapeutic agents, such as other immunotherapeutic agents, cytokine and lymphokine muteins, as well as immunotoxins and small molecule chemotherapeutic agents. Such agents include, without limitation, interleukins, including IL-1, IL-2, IL-3, IL-4, IL-5, IL-12 and muteins of these molecules; interferons, such as but not limited to IFN-α, IFN-β, IFN-γ and muteins thereof; GM-CSF and muteins of GM-CSF; tumor necrosis factors, such as TNF-α and TNF-β and muteins of these molecules; anti-ganglioside antibodies, cyclosporin A, cyclophosphamide, mitomycin C, FK973, monocrotaline pyrrole and cytosine arabinoside and muteins of these molecules; and various immunotoxins.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

A. Media and Reagents:

PBS (without Ca/Mg), ice cold;

Medium 200 (Cascade Biologics, Portland, Oreg.);

Medium 200PRF (Cascade Biologics, Portland, Oreg.);

LSGS: Low Serum Growth Supplement (50×). (Cascade Biologics, Portland, Oreg.). Final concentrations of the components in the supplemented medium were: fetal bovine serum, 2% v/v; hydrocortisone, 1 μg/ml; human epidermal growth factor, 10 ng/ml; human epidermal growth factor, 10 ng/ml; basic fibroblast growth factor, 3 ng/ml and heparin, 10 μg/ml;

PSA solution (Cascade Biologics, Portland, Oreg.): final concentration in complete medium contained 100 U/ml Penicillin G, 100 μg/ml Streptomycin sulfate, and 0.25 μg/ml Amphotericin B;

Trypsin (0.25%)/EDTA (0.1%) (Cellgro, Herndon, Va.);

FITC-albumin (50 mg) (Sigma, St. Louis, Mo.);

FITC-BSA stock solution (20×). FITC-BSA was suspended in 2.5 ml complete Medium 200.

Human AB serum (SeraCare Life Sciences, Oceanside, Calif.)

Human AB medium (500 mL)

| | | |
|---|---|---|
| RPMI (Phenol Red free) | 426.5 | ml |
| Human AB-heat inactivated* | 50 | ml |
| Pen/Strep (final 100 μg/ml) | 5 | ml |
| HEPES (1M stock, 25 mM final) | 12.5 | ml |
| L-glutamine (100 × stock, final 2 mM) | 5 | ml |
| Fungizone (250 μg/ml stock, 0.5 μg/ml final) | 1 | ml |

Store media up to 4 weeks at 4° C.
*Thaw serum overnight at 4° C., Heat inactivate 45 min at 56° C.

RPMI: ice cold, serum-free;

PBS/EDTA: 5.7 ml 0.5 M EDTA added to 500 ml PBS (without Ca/Mg), final pH 7.2;

Trypan blue stain (Invitrogen Life Technologies, Carlsbad, Calif., or equivalent 0.4% solution);

10% saponin stock: 2.5 g saponin in 25 ml PBS, stored at 4° C.;

Complete Medium 200:

| | |
|---|---|
| Medium 200/Medium 200PRF | 500 ml |
| LSGS | 10 ml |
| PSA solution | 1 ml |

B. Cell Culture:

HUVEC (Human Umbilical Vein Endothelial Cells) were obtained from Cascade Biologics, Portland, Oreg. Each vial contained≧5×10⁵ cells. The vials were thawed by dipping into 37° C. water. Cells were diluted to 10 ml using Complete Medium 200 and the number of viable cells per ml was determined using Trypan blue counting. Complete Medium 200 was then used to dilute the contents of the vial to a concentration of 1.25×10⁴ viable cells/ml. 5 ml or 15 ml of cell suspension was added to 25 cm² or 75 cm² flasks, respectively, and the media swirled in the flasks to distribute the cells. Cultures were incubated in a humidified 5% CO² incubator at 37° C. After 24-36 hours, the medium was changed to fresh supplemented Medium 200 and everyday thereafter until the culture was approximately 80% confluent. This took approximately 5-6 days.

The HUVEC were then subcultured as follows. Culture medium was removed, trypsin/EDTA solution was added to the flask, cells were dislodged by tapping and aspirated off and Complete Medium 200 added to transfer the cells to a sterile 15 ml conical tube. Cells were centrifuged at 1000 rpm for 10 min., counted and seeded at 2.5×10³ viable cells/cm².

Cells were cryopreserved in freeze medium (90% FBS, 10% DMSO) during passage 2 or 3 and stored in liquid nitrogen for future use.

### EXAMPLE 1

### In Vitro Assay for Screening IL-2 Muteins

In order to test the ability to predict patient tolerability of IL-2 muteins, the following assay was conducted. Two IL-2 muteins with improved tolerability were used in the assay as proof of principle. These muteins were F42E and Y107R substitution mutants. See, commonly owned, copending U.S. Provisional Application Ser. No. 60/550,868, filed Mar. 5, 2004. These muteins also maintain effector function in in vitro and in vivo models in terms of NK and T cell proliferation, as well as NK/LAK/ADCC activity. In addition, Proleukin®, Chiron Corporation, Emeryville, Calif. was used as a representative IL-2 molecule that can cause VLS. The IL-2 in this formulation is a recombinantly produced, unglycosylated human IL-2 mutein, called aldesleukin, which differs from the native human IL-2 amino acid sequence in having the initial alanine residue eliminated and the cysteine residue at position 125 replaced by a serine residue (referred to as des-alanyl-1, serine-125 human interleukin-2). This IL-2 mutein is expressed in E. coli, and subsequently purified by diafiltration and cation exchange chromatography as described in U.S. Pat. No. 4,931,543. The IL-2 formulation marketed as Proleukin® is supplied as a sterile, white to off-white preservative-free lyophilized powder in vials containing 1.3 mg of protein (22 MIU).

Lymphokine-activated killer (LAK) cells, for use in the assay were prepared as follows. Whole blood was collected from normal donors and placed into Vacutainer CPT tubes (ACDA, Becton Dickinson, Franklin Lakes, N.J.). PBMCs were separated according to CPT manufacturer specifications. Briefly, tubes were inverted to mix, centrifuged at 1500-1800×g for 20 min., the buffy coat removed, placed into conical vial and washed with PBS 2% FBS (maximum 300×g, 15 min.). The supernatant was removed and the cells washed twice. PBMC were suspended in RPMI-10AB medium and enumerated via Trypan blue dye exclusion using a hemacytometer. PBMC were suspended at 1.5×10⁶ cells/ml in RPMI-10AB (Phenol Red-free). 1 ml of the PBMC suspension was added to each well of a 24-well tissue culture plate and 1 ml/well of 37° C. RPMI-10AB medium containing 50 nm of the desired IL-2 mutein was also added. The plate was covered and incubated for 3 days in a humidified 37° C., 5% CO₂ incubator after which time the plates were placed on ice for approximately 30 min., supernatants removed and centrifuged at 300×g, 4° C. for 5 min. and saved in 50 ml conical tubes on ice. 1 ml ice-cold PBS/EDTA was added to each well and incubated for 20 min. Adherent cells were removed and added to the tubes on ice. 1 ml cold PBS was added to each well. The 50 ml conical tubes were centrifuged for 5 min., 300×g, 4° C. In the meantime, wells were washed and the wash was placed in 50 ml conical tubes on ice. Supernatants were decanted, the cell pellet suspended in 12 ml cold RPMI and added to the PBS wash. This was centrifuged again for 5 min., 300×g, 4° C. Supernatants were again decanted and the cell pellet suspended and centrifuged as above. The washed cells were suspended in cold complete RPMI and counted by Trypan blue exclusion.

600 μl Complete Medium 200PRF was added to the lower chamber of collagen-coated transwells (Transwell-COL™, collagen coated PTFE membrane, Costar (Corning, Inc., Corning, N.Y.), HUVEC, passaged less than 5 times, as described above, were removed from the flasks and suspended at 1.0×10⁶ cells/ml in Complete Medium 200PRF. 100 μl of the HUVEC suspension was added to the upper chamber of the transwells. Control transwells with media but no cells were also set up to measure maximum transfer of FITC-BSA across the collagen membrane. The transwells were incubated for 3 days in a humidified 5% CO² incubator at 37° C. to establish confluent monolayers. On day 4, the monolayers were stained with crystal violet (Sigma, St. Louis, Mo.; 2.3% w/v, ammonium oxalate 0.1% w/v, ethyl alcohol, SD3A 20% v/v) to check for confluence. The assay proceeded when monolayers were 90% confluent.

The Complete Medium 200 was removed completely from the upper chamber of the transwell and 100 μl Complete Medium 200 with FITC-BSA (1 mg/ml) was added to both the test and control transwells. Plates were covered with aluminum foil and sampled at 30 min. by removing 10 μl of sample from the lower chamber of the transwells to a black 96-well plate with a clear bottom. 10 μl Complete Medium was then added back to the lower chamber. 40 μl of Complete Medium 200 was added to the black 96-well plate and mixed. The samples were read using IL-2 mutein-fluorescein (485/575 nm, 1.0 s). Transwells were reallocated based on the fluorescent intensity readout, which for 90% or higher confluent monolayers, should be lower than 6,000. After 3 hours of incubation, the plates were again sampled and read as above. The 3 hour intensity readout was considered baseline or time 0.

The FITC-BSA was removed from the upper chamber and 100 μl of the test compounds with FITC-BSA (1 mg/ml) was added to the upper chamber: (1) IL-2 muteins F42E, Y107R or Proleukin® (25 nM); (2) 3 day 25 nM IL-2-mutein-stimulated PBMC supernatant; (3) 3 day 25 nM IL-2-mutein-stimulated LAK cells; and (4) 3 day 25 nM IL-2-mutein-stimulated LAK cells in supernatant. Additionally, 0.05% saponin was used as a positive control as saponin destroys the monolayers and Complete Medium 200 was used as a medium control. The plate was covered with aluminum foil and incubated at 37° C. Samples were obtained after incubation for 3 hours and 22 hours with the test compound and fluorescence read as described above.

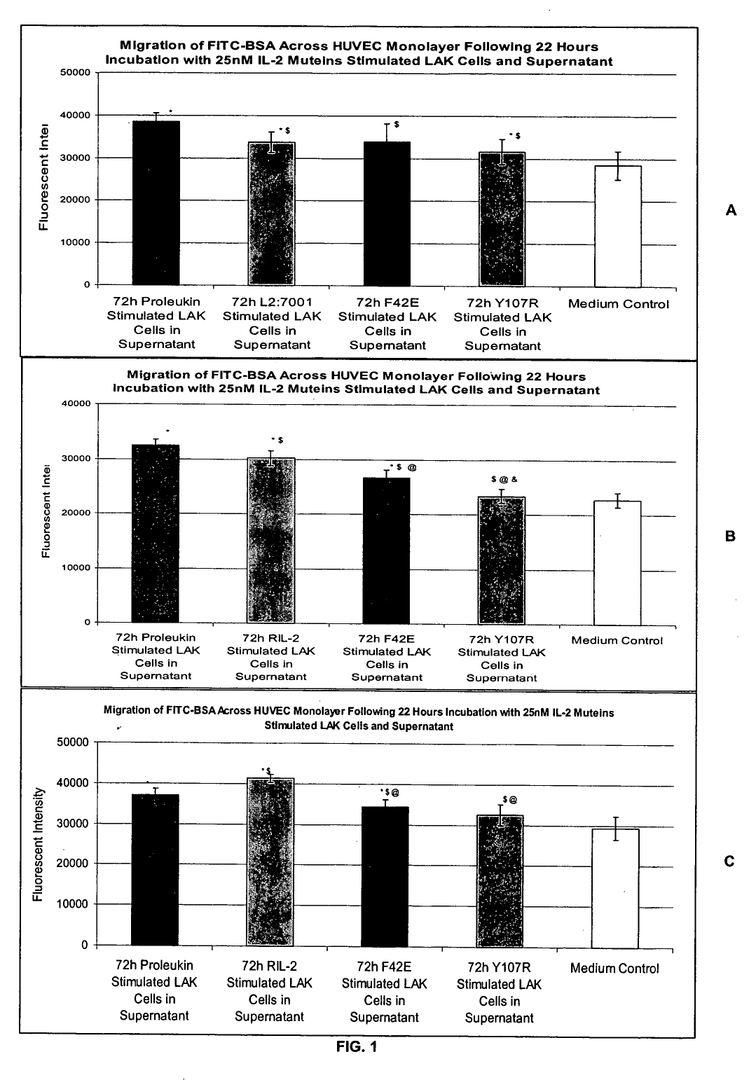

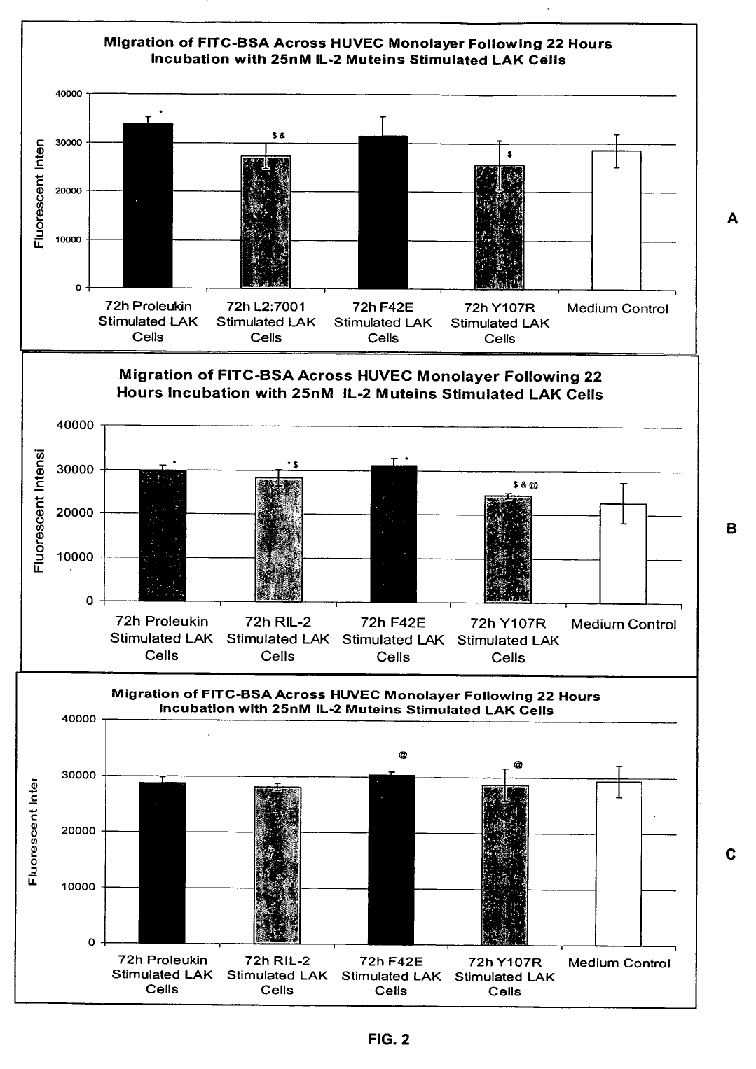

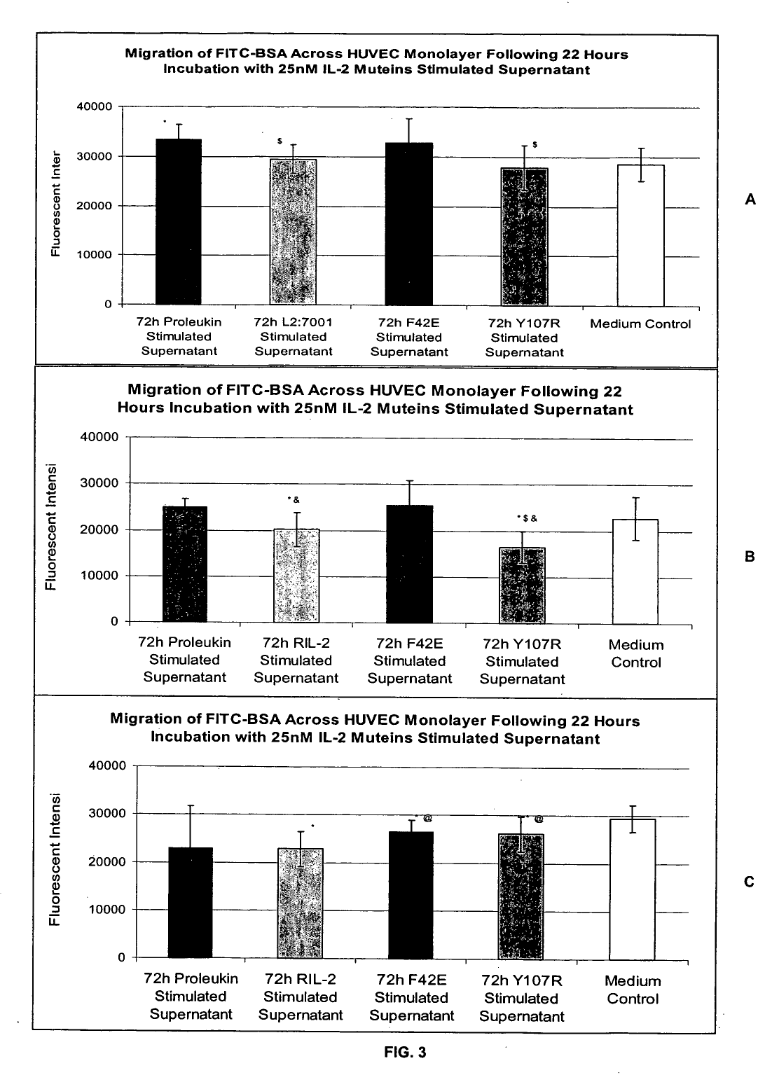

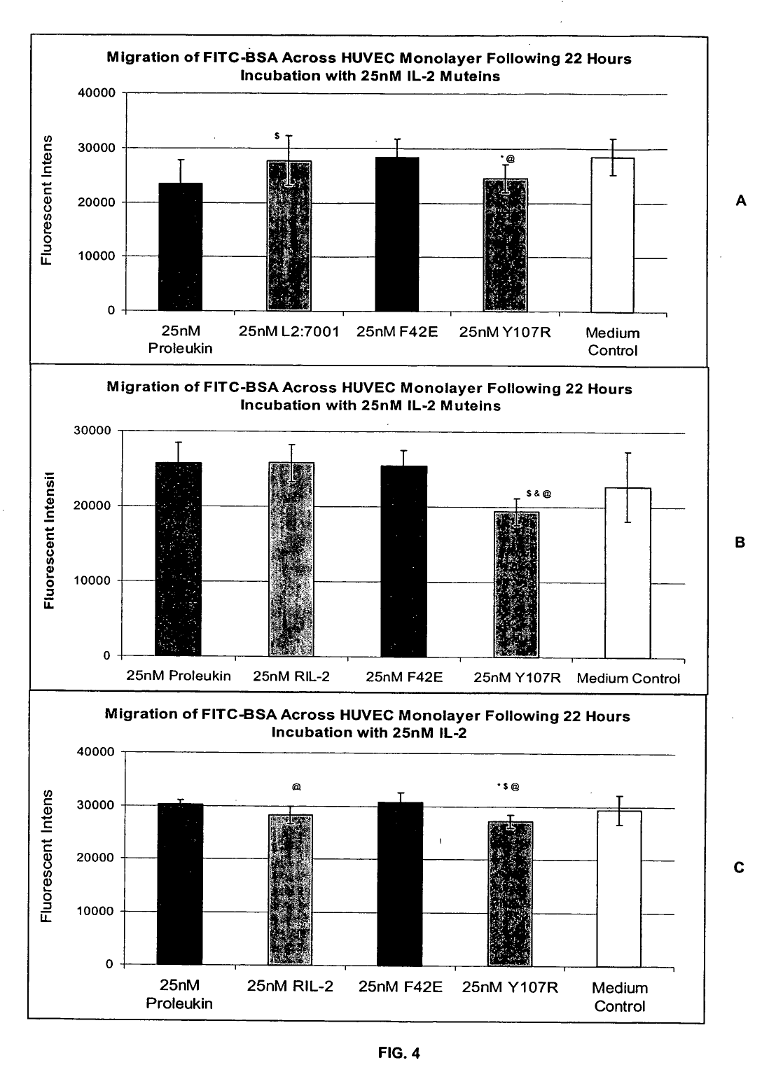

Results are presented in FIGS. 1-4. The data shown is from 3 independent experiments, n=5 or 6 per test condition, from 3 different normal human donors provided in the figures. As seen in FIGS. 1A-1C, Proleukin®-stimulated PBMC (LAK) and supernatants resulted in significant damage to endothelial cell monolayers. In 2 of the 3 donors tested, the F42E and Y107R muteins displayed significantly reduced VLS as compared to Proleukin®. As shown in FIGS. 2A-2C, in 2 of 3 donors tested, Proleukin® induced LAK cells alone showed significantly increased VLS over medium control samples, but no significant difference between medium controls and F42E or Y107R were observed. As seen in FIGS. 3A-3C, culture supernatants from IL-2-induced PBMC alone were not sufficient to significantly induce VLS syndrome in vitro. As shown in FIGS. 4A-4C, neither Proleukin® or F42E and Y107R alone induced damage to the EC monolayer directly. As seen in FIGS. 1-4, there were no significant differences between F42E and Y107R alone, F42E- and Y107R-stimulated supernatant, F42E- and Y107R-stimulated LAK cells alone and the medium control.

Accordingly, the in vitro model of VLS that measures permeability of FITC-BSA across confluent monolayers of endothelial cells was valid and consistent.

Thus, novel in vitro test systems for predicting patient tolerability to IL-2 muteins are disclosed. Although preferred embodiments of the subject invention have been described in some detail, it is understood that obvious variations can be made without departing from the spirit and the scope of the invention as described in the appended claims.

1. An in vitro method for predicting tolerability or intolerability by a patient to a selected therapeutic agent, said method comprising:
(a) providing a confluent monolayer of endothelial cells attached to an adherence substrate;
(b) contacting said monolayer with
(i) said selected therapeutic agent, or a preparation of lymphokine-activated killer (LAK) cells wherein said LAK cells are produced by activating peripheral blood mononuclear cells using said therapeutic agent, or the supernatant from said LAK cells, and
(ii) a detectably labeled macromolecule, wherein said detectably labeled macromolecule is substantially retained by said confluent monolayer when said monolayer is intact;
(c) incubating said monolayer from step (b) for a period of time and under conditions that allow for said detectably labeled macromolecule to pass through said confluent monolayer and said adherence substrate if the integrity of said monolayer is disrupted; and
(d) detecting macromolecule that passes through said confluent monolayer and said adherence substrate as an indication of tolerability or intolerability by a patient to said therapeutic agent.

2. The method of claim 1, wherein said therapeutic agent is an immunotherapeutic agent, an immunotoxin or a small molecule chemotherapeutic agent.

3. The method of claim 2, wherein said immunotherapeutic agent is an interleukin-2 (IL-2) mutein.

4. The method of claim 1, wherein said adherence substrate comprises a collagen matrix.

5. The method of claim 1, wherein said endothelial cells are human umbilical vein endothelial cells (HUVEC).

6. The method of claim 1, wherein said detectably labeled macromolecule is a detectably labeled albumin.

7. The method of claim 6, wherein said detectably labeled albumin is a labeled bovine serum albumin (BSA).

8. The method of claim 7, wherein said BSA is fluorescently labeled.

9. The method of claim 8, wherein said fluorescent label is FITC.

10. An in vitro method for predicting tolerability or intolerability by a patient to an interleukin-2 (IL-2) mutein, said method comprising:
(a) providing a confluent monolayer of endothelial cells attached to an adherence substrate;
(b) contacting said monolayer with
(i) a preparation of lymphokine-activated killer (LAK) cells, wherein said LAK cells are produced by activating peripheral blood mononuclear cells using said IL-2 mutein, and
(ii) a detectably labeled macromolecule, wherein said detectably labeled macromolecule is substantially retained by said confluent monolayer when said monolayer is intact;
(c) incubating said monolayer from step (b) for a period of time and under conditions that allow for said detectably labeled macromolecule to pass through said confluent monolayer and said adherence substrate if the integrity of said monolayer is disrupted; and
(d) detecting macromolecule that passes through said confluent monolayer and said adherence substrate as an indication of tolerability or intolerability by a patient to said IL-2 mutein.

11. The method of claim 10, wherein said adherence substrate comprises a collagen matrix.

12. The method of claim 10, wherein said endothelial cells are human umbilical vein endothelial cells (HUVEC).

13. The method of claim 10, wherein said detectably labeled macromolecule is a detectably labeled albumin.

14. The method of claim 13, wherein said detectably labeled albumin is a labeled bovine serum albumin (BSA).

15. The method of claim 14, wherein said BSA is fluorescently labeled.

16. The method of claim 15, wherein said fluorescent label is FITC.

17. An in vitro method for predicting tolerability or intolerability by a patient to an interleukin-2 (IL-2) mutein, said method comprising:
(a) providing a confluent monolayer of human umbilical vein endothelial cells (HUVEC) attached to an adherence substrate comprising a collagen matrix;
(b) contacting said monolayer with
(i) a preparation of lymphokine-activated killer (LAK) cells, wherein said LAK cells are produced by activating peripheral blood mononuclear cells using said IL-2 mutein, and
(ii) a fluorescently labeled albumin;
(c) incubating said monolayer from step (b) for a period of time and under conditions that allow for said fluorescently labeled albumin to pass through said confluent monolayer and said adherence substrate if the integrity of said monolayer is disrupted; and
(d) detecting fluorescently labeled albumin that passes through said confluent monolayer as an indication of tolerability or intolerability by a patient to said IL-2 mutein.

18. The method of claim 17, wherein said fluorescently labeled albumin is a labeled bovine serum albumin (BSA).

19. The method of claim 18, wherein said fluorescent label is FITC.
